(19) United States
(12) Patent Application Publication
(10) Pub. No.: US 2024/0260930 A1
(43) Pub. Date: Aug. 8, 2024

(54) DEVICE FOR LOCATING A BLOOD VESSEL

(71) Applicant: Arterya, Caen (FR)

(72) Inventors: Lucile Derly, Caen (FR); Nicodème Pellier, Beauchamps (FR); Benjamin Calloch, Fresney Le Puceux (FR); Benoit Agnus, Villons Les Buissons (FR)

(21) Appl. No.: 18/565,588

(22) PCT Filed: May 20, 2022
(86) PCT No.: PCT/EP2022/063725

§ 371 Date: Nov. 30, 2023

(30) Foreign Application Priority Data
Jun. 3, 2021 (FR) FR2105855
Sep. 9, 2021 (FR) FR2109449

(51) Int. Cl. A61B 8/08; A61B 8/00

(52) U.S. Cl. CPC A61B 8/085; A61B 8/4227; A61B 8/461

(57) ABSTRACT

The invention relates to a device for locating a blood vessel of a limb of a living being, configured to be arranged substantially opposite a skin surface covering the blood vessel of said limb. According to the invention, the device comprises at least one acoustic wave transmitter configured to transmit acoustic waves, hereinafter transmitted acoustic waves, in the limb through the skin surface, at least one acoustic wave receiver configured to receive the acoustic waves reflected by the limb, hereinafter reflected acoustic waves, a module for measuring a parameter of the reflected acoustic waves or a difference in value of a parameter between the reflected acoustic waves and the transmitted acoustic waves, and a module for determining the location of the blood vessel depending on said parameter or said difference in value of said parameter.

### TECHNICAL FIELD OF THE INVENTION

The present invention generally relates to techniques for locating blood vessels, and in particular to the corresponding devices.

### TECHNICAL BACKGROUND

Blood gas analysis (or blood gasometry) is called “blood gas”. During such an analysis, the practitioner measures the acidity of the blood and the amounts of oxygen and carbon dioxide in the blood. The blood is preferentially taken from an artery.

The examination makes it possible to evaluate pulmonary gas exchange and detect changes in oxygen and carbon dioxide concentrations in the arterial blood, particularly in the blood flowing to the tissues. In fact, when blood moves through the lungs, for example, it is enriched in oxygen and depleted in carbon dioxide.

Blood gas analysis can also be used to evaluate a patient's acid-base balance.

As part of the examination, blood is drawn from an artery. In general, this involves the radial (wrist), humeral (upper arm) or femoral (groin) artery. Once the sample has been collected, a gauze or cotton pad must be applied and the puncture site compressed firmly for a few minutes.

It would therefore be advantageous to pinpoint the location of the artery by non-invasive means prior to drawing blood and to provide the practitioner with a visual indication of the artery's location to facilitate the blood draw.

It would be particularly useful to display the location of the artery over a sufficient length to enable the practitioner to determine the direction of the artery so that the needle or stylet can be positioned to pierce the artery generally or approximately along its axis. It is generally desirable to intersect the artery at an angle comprised between approximately 30 degrees and 45 degrees. Once the artery is suitably located, the practitioner can then position the needle at an appropriate angle for insertion into the blood vessel.

Systems such as those used to locate a blood vessel have been described in the prior art. For example, U.S. patent application No. US2018325448A1 discloses an artery locating device that comprises a sensor array configured to be attached to the surface of the skin covering a target artery.

The sensor array is an array of detectors, for example pressure detectors configured to generate signals that are sensitive to pressure or pressure changes. A display device is arranged on the sensor array. A control circuit is configured to receive signals generated by the sensor array, to identify from the received signals periodic pressure pulses that have a frequency within a predetermined frequency range corresponding to a pulsatile frequency and that define an elongated path through at least part of the sensor array, and to display an image on the display device that covers the elongated path through the sensor array, such that the display shows a projection of the two-dimensional position of the artery below the display. However, such a locating device based on a sensor array is relatively cumbersome and is not precise enough to detect arteries under all conditions in which the device is used.

### SUMMARY OF THE INVENTION

Based on this problem, the present invention therefore aims to develop a device for locating a blood vessel of the type previously indicated in order to guarantee greater precision. According to certain embodiments, the invention also aims to provide such a locating device that is less cumbersome than certain prior art devices. According to certain embodiments, the invention also aims to provide such a locating device that indicates to the user, who may be a caregiver or practitioner, a point at which to perform an operation (such as a blood draw). With regard to devices for locating blood vessels, the task on which the invention is based is addressed by the subject-matter of the present invention, with advantageous developments of the locating device according to the invention being described hereinafter.

Therefore, the invention relates in particular to a device for locating a blood vessel of a limb of a living being, configured to be arranged substantially opposite a skin surface covering the blood vessel of said limb, characterized in that the device comprises at least one acoustic wave transmitter configured to transmit acoustic waves, hereinafter transmitted acoustic waves, in the limb through the skin surface, at least one acoustic wave receiver configured to receive the acoustic waves reflected by the limb, hereinafter reflected acoustic waves, a module for measuring a parameter of the reflected acoustic waves or a difference in value of a parameter between the reflected acoustic waves and the transmitted acoustic waves, and a module for determining the location of the blood vessel depending on said parameter or said difference in value of said parameter.

The locating device according to the invention can be used to locate any type of blood vessel, such as veins, capillaries or arteries, for example. It can be advantageously used on human beings but can also be used on animals (veterinary applications).

Preferentially, the locating device according to the invention is used to locate an artery as part of an arterial blood draw.

Preferentially, the measuring module is a module for measuring the phase shift between the reflected acoustic waves and the transmitted acoustic waves and in that the module for determining the location of the blood vessel takes account of said measured phase shift.

Preferentially, the measuring module is a module for measuring the amplitude of the reflected acoustic waves and in that the module for determining the location of the blood vessel takes account of said amplitude.

Preferentially, the measuring module is a module for measuring the difference in frequency between the reflected acoustic waves and the transmitted acoustic waves and in that the module for determining the location of the blood vessel takes account of said difference in frequency.

Preferentially, the device comprises a first acoustic wave transmitter and a first acoustic wave receiver located in a first area of the device and a second acoustic wave transmitter and a second acoustic wave receiver located in a second area of the device.

Preferentially, the device comprises a first matrix of acoustic wave transmitters and receivers located in a first area of the device, and a second matrix of acoustic wave transmitters and receivers located in a second area of the device.

Preferentially, the means for measuring a parameter or a difference in value of a parameter comprise means for measuring a first parameter value or parameter value difference in the first area and means for measuring a second parameter value or parameter value difference in the second area and in that the means for determining the location take account of a first location determined from the first parameter value or parameter value difference and of a second location determined from the second parameter value or parameter value difference.

Preferentially, each acoustic wave transmitter and/or receiver can be respectively reconfigured as an acoustic wave receiver and/or transmitter.

Preferentially, the acoustic wave transmitters and receivers comprise piezoelectric elements. Preferentially, the module for measuring a parameter or a difference in value of a parameter comprises a filter and an amplifier.

Preferentially, the device comprises a module for displaying the location of the blood vessel, comprising a first laser and a second laser whose beams intersect at a point on the surface of the skin, said point being indicative of the location of the blood vessel.

The invention also relates to a strap adapted to be fastened to a limb of a living being, characterized in that it comprises the locating device according to the invention.

Preferentially, the blood vessel is an artery, the living being is a human being and the limb is a wrist.

Preferentially, the strap comprises a matrix made of silicone.

The invention further relates to a method for manufacturing a device for locating a blood vessel and a strap comprising such a device, in particular a device for locating a blood vessel and a strap of the type according to the invention.

### BRIEF DESCRIPTION OF THE FIGURES

Other features and advantages of the invention will become apparent from the following detailed description, which will be understood by referring to the appended drawings in which:

FIG. 1 shows a schematic top view of a strap comprising a device for locating a blood vessel according to an embodiment of the invention;

FIG. 2 shows a schematic view of the underside of the strap in FIG. 1;

FIG. 3 shows a schematic cross-sectional view of the strap in FIG. 1, seen from the side;

FIG. 4 shows a schematic cross-sectional view (perpendicular to the strap axis) of the strap shown in FIG. 1;

FIG. 5 shows a schematic view of the strap in FIG. 1 fastened to a wrist, and the path of the transmitted and reflected acoustic waves;

FIG. 6 shows a graph illustrating the amplitude of the phase shift as a function of time over the course of a scan of a matrix of piezoelectric disks in the strap in FIG. 1, in a first embodiment of the invention.

FIG. 7 shows a block diagram of the electronic circuit of the strap shown in FIG. 1.

### DETAILED DESCRIPTION OF THE INVENTION

Various aspects of various embodiments of a device for locating a blood vessel and of a strap comprising such a device are described in greater detail below, with reference to the accompanying drawings.

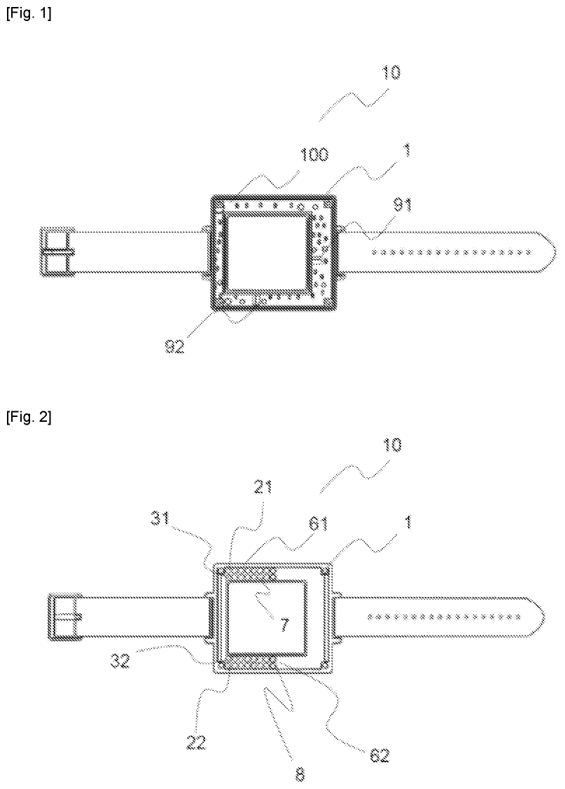

FIGS. 1 to 4 show a schematic view of an exemplary embodiment of a strap 10 comprising a device 1 for locating a blood vessel according to the invention. In this embodiment of the invention, the strap is used to detect and locate the radial artery 11 in the wrist 12 of a human patient.

Of course, according to other embodiments, the strap or even the locating device can be used to locate any blood vessel such as capillaries or veins or arteries in any living being such as human beings or animals. Likewise, they can be used for such detection in any limb such as, for example, the wrist, arm, groin, and leg or hand or foot.

In this embodiment of the invention, the strap is used by a caregiver or practitioner as part of an arterial blood draw to perform an analysis of the patient's blood gas. Therefore, in this example, the target artery is the radial artery 11 in the patient's wrist 12.

Preferentially, the strap 10 is attached to the patient's wrist 12 such that the device 1 is positioned opposite the inner face of the wrist 12.

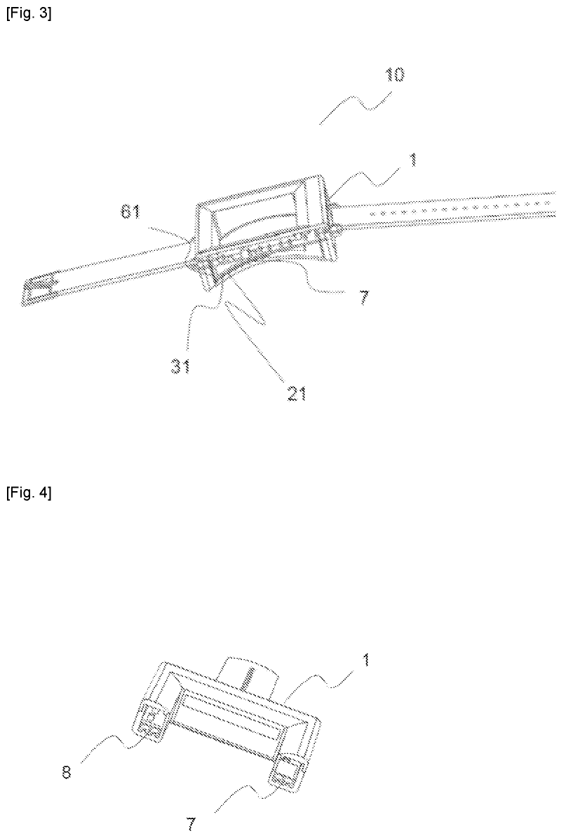

More specifically, FIG. 1 shows a schematic top view of the strap 10. FIG. 2 shows a schematic view of the underside of the strap 10. FIG. 3 shows a schematic cross-sectional side view of the strap 10. FIG. 4 shows a schematic cross-sectional view (perpendicular to the strap 10 axis) of the strap 10.

The locating device 1 is configured to be positioned substantially opposite a skin surface covering the wrist artery. According to a preferential embodiment of the invention, the locating device 1 is configured to be positioned substantially in contact with a skin surface covering the wrist artery. However, according to variants of this embodiment of the invention, the locating device can be designed to be positioned at a distance (for example, a few millimeters or a few centimeters) from the skin surface.

According to an embodiment of the invention, the device 1 is designed to include two strands attached to the device by means of a joint in the manner of a watch strap. The two strands can be made of silicone, leather, nylon, or any other synthetic material. The two strands can be fastened to each other at their free ends by any fastening mechanism (for example, a snap or a buckle with a metal pin, etc.). Of course, a strap according to the invention can also comprise a single strand (for example a matrix) made of silicone with a receptacle for holding the device 10.

The device 1 comprises at least one acoustic wave transmitter 2, 21, 31 configured to transmit acoustic waves (hereinafter transmitted acoustic waves) in the limb through the skin surface, at least one acoustic wave receiver 3, 31, 32 configured to receive the acoustic waves reflected by the wrist 12 (hereinafter reflected acoustic waves).

As illustrated in FIGS. 2 to 4, the device 1 comprises a first matrix 7 of acoustic wave transmitters 21 and receivers 31 located in a first area 61 of the device 1, and a second matrix 8 of acoustic wave transmitters 22 and receivers 32 located in a second area 62 of the device 1.

Of course, according to variants of the invention, the device can comprise any number of acoustic wave transmitters and receivers in the first area 61, whether organized in a matrix or not and it can also comprise any number of acoustic wave transmitters and receivers in the second area 62, whether organized in a matrix or not. For example, the device comprises a first acoustic wave transmitter and a first acoustic wave receiver located in the first area 61 of the device 1 and a second acoustic wave transmitter and a second acoustic wave receiver located in the second area 62 of the device.

For example, when viewed from the top, the device 1 has a substantially square or rectangular shape. Preferentially, the distance between the first area 61 where the first matrix 7 is located and the second area 62 where the second matrix 8 is located is less than 2 cm and even preferentially less than 1.5 cm such that the artery can be considered substantially straight between the first matrix 7 and the second matrix 8.

According to a preferential embodiment, each acoustic wave transmitter 2, 21, 22 and receiver 3, 31, 32 can be respectively reconfigured as an acoustic wave receiver and/or transmitter. Of course, according to variants of this preferential embodiment, only part of the transmitters and/or receivers (for example at least one of them) can be reconfigured. According to yet another variant, none of the receivers or transmitters can be reconfigured.

For example, each of the acoustic wave transmitters and receivers comprises a piezoelectric element, for example a piezoelectric disk with a diameter preferentially comprised between 0.5 mm and 1 cm and even more preferentially comprised between 1 mm and to 3 mm. For example, each of the piezoelectric disks has a diameter of 1 mm. Of course, the invention can be carried out using piezoelectric disks with diameters with any other value. Moreover, any other form of piezoelectric element can be used within the scope of the invention. In addition to piezoelectric solutions, other types of transmitters and receivers can also be used within the scope of the invention.

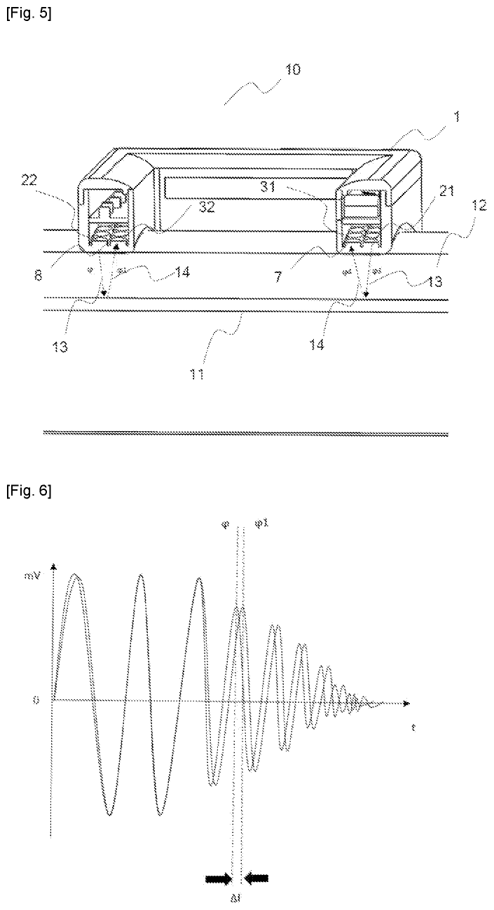

The device 1 on the strap 10 also comprises means of controlling the transmitters and receivers. For example, these control means are in the form of an electronic circuit 100. Thus, according to a preferential embodiment of the invention, the electronic circuit can power some of the transmitters and receivers of the first matrix 7 and second matrix 8 according to a set sequence. For example, referring to FIG. 2 and FIG. 5 which show a schematic view of the strap 10 fastened to the wrist 12 and the path of the transmitted acoustic waves 13 and reflected acoustic waves 14, such a sequence can comprise the simultaneous performance of the following four steps:
Configure the first piezoelectric disk of the first matrix as a transmitter, starting from the left on the outer row of the first matrix (the row of piezoelectric disks that is farthest from the center of the device 10)
Configure the first piezoelectric disk of the first matrix as a receiver, starting from the left on the inner row of the first matrix (the row of piezoelectric disks that is closest to the center of the device 10)
Configure the first piezoelectric disk of the second matrix as a transmitter, starting from the left on the inner row of the second matrix (the row of piezoelectric disks that is closest to the center of the device 10)
Configure the first piezoelectric disk of the second matrix as a receiver, starting from the left on the inner row of the second matrix (the row of piezoelectric disks that is farthest from the center of the device 10)

Then, simultaneously perform the same four steps with the second piezoelectric disk in each row of each matrix, and so on, so as to horizontally scan all the piezoelectric disks in the first and second matrices.

Such a scan makes it possible to measure the reflected acoustic waves 14 from the wrist 12 along the entire length of the piezoelectric disk matrices.

The length of the first and second matrices is identical, and the spacing between the disks in each row is also identical. In this way, each row of each matrix comprises the same number of piezoelectric disks. For example, the length of the first matrix 7 and second matrix 8 is planned in the device 1 so that each matrix in the device is directly above the artery 11 in the wrist 12.

The electronic circuit 100 comprises a module for measuring a parameter of the reflected acoustic waves 14 or a difference in value of a parameter between the reflected acoustic waves 14 and the transmitted acoustic waves 13. The electronic circuit 100 also comprises a module for determining the location of the blood vessel as a function of said parameter or said difference in the value of said parameter.

Preferentially, the module for measuring the parameter of the reflected acoustic waves 14 or a difference in value of a parameter between the reflected acoustic waves 14 and the transmitted acoustic waves 13 comprises a filter and an amplifier.

Preferentially, the means for measuring a parameter or a difference in value of a parameter comprise means for measuring a first parameter value or parameter value difference in the first area and means for measuring a second parameter value or parameter value difference in the second area.

According to a first embodiment of the invention, the measuring module is a module for measuring the phase shift between the reflected acoustic waves and the transmitted acoustic waves.

According to a second embodiment of the invention, the measuring module is a module for measuring the amplitude of the reflected acoustic waves.

According to a third embodiment of the invention, the measuring module is a module for measuring the difference in frequency between the reflected acoustic waves and the transmitted acoustic waves.

FIG. 6 shows a graph illustrating the amplitude of the phase shift Af to mV as a function of time t over the course of a scan of a matrix of piezoelectric disks in the strap 10 in the aforementioned first embodiment. The phase shift Af is equal to the phase difference Phi 1-Phi between the reflected waves 14 and the transmitted waves 13.

Thus, for example, when scanning the matrices of piezoelectric disks used by the electronic circuit described above, the phase shift measuring means will measure the phase shift between the transmitted waves 13 and the reflected waves 14 for each pair of piezoelectric disks on both rows of each matrix and make it possible to obtain the graph as illustrated in FIG. 6, which represents the measurement of the phase shift as a function of time over the course of a scan of one of the first matrix 7 and second matrix 8, for example the second matrix 8.

When the transmitted acoustic waves reach the artery 11, the phase shift between the transmitted waves and the reflected waves will reach a maximum value that is characteristic of the presence of the artery. Thus, from the measured phase shift as shown in FIG. 6, the electronic circuit of the device can identify the maximum phase shift value that is characteristic of the presence of the artery in the wrist under the piezoelectric disk pair.

In the second embodiment, the amplitude of the reflected waves is measured instead of the phase shift and, similarly, the maximum value of the amplitude is characteristic of the presence of the artery.

In the third embodiment, the difference in frequency between the reflected and transmitted waves is measured instead of the phase shift and, similarly, the maximum value of the difference in frequency is characteristic of the presence of the artery.

The electronic circuit 100 also comprises a module for determining the location of the artery as a function of the phase shift measured in the aforementioned first embodiment (or as a function of the amplitude of the reflected acoustic waves in the aforementioned second embodiment or even as a function of the difference in frequency between the reflected waves and the transmitted waves in the aforementioned third embodiment).

This module will therefore identify the maximum phase shift measured by the phase shift measurement module in the first embodiment (or the maximum amplitude of the reflected acoustic waves in the second embodiment or the maximum difference in frequency in the third embodiment) when scanning over each of the first and second matrix, which enables it to determine which piezoelectric disk pair corresponds to the minimum in each of the first matrix 7 and second matrix 8.

This enables it to locate the artery at two points: one at the first matrix and the other at the second matrix. Then, for example, the module for determining the location of the artery 11 makes a linear interpolation between the two points and determines a point situated in the middle of the two aforementioned points on the straight line running through these two points. This midpoint is representative of the location of the artery 11.

The device 10 also comprises a module for displaying the location of the artery, comprising a first laser 91 and a second laser 92 that are controlled by the electronic circuit 100 and whose beams intersect at a point on the surface of the skin, said point being indicative of the location of the blood vessel. Preferentially, the first laser 91 and second laser 92 are provided so as to be translationally movable respectively along a first and a second side of the device for displaying the location of the artery. For example, each of the first laser 91 and second laser 92 are mounted on first and second linear actuators provided on a board holding the electronic circuit. The actuators can be controlled by the electronic circuit and, for example, by the module for determining the location of the artery as a function of the measured phase shift (or of the measured amplitude, or even of the measured difference in frequency). Preferentially, the first and second sides are orthogonal.

The display module controls the movement of the first laser 91 and second laser 92 so that their respective beams intersect at the point representing the location of the artery 11.

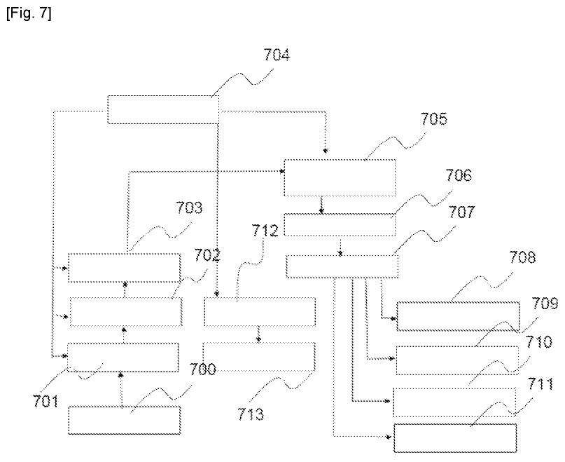

FIG. 7 shows a block diagram of the electronic circuit of the strap 10 in FIG. 1.

An acoustic signal 700 (reflected on the wrist) is received by a piezoelectric disk configured as a receiver 701. The electric signal generated as a result by the piezoelectric disk (receiver) is then transmitted to a noise filter 702, which in turn transmits the filtered signal to an amplifier 703.

The noise filter 702 is, for example, a third-order active high-pass filter which, for example, has a gain of 24 dB (obtained by the following formula: Gain=33000/1500+22000/9100˜=24 dB).

The amplifier 703 is, for example, an AD847 amplifier that has a gain per bandwidth product of about 50 MHz. The amplifier 703 then transmits the amplified signal to a data processing module 705 (which comprises the aforementioned module for measuring a parameter or parameter difference and the module for determining the location). The data processing module 705 transmits a signal to a control module 706 that controls a power supply module 707 which in turn powers a motor drive for the actuator of the first laser 91, a motor drive for the actuator of the second laser 92, as well as the first laser 91 and the second laser 92. A power supply 704 powers the piezoelectric transmitter and receiver disks, the noise filter 702 and the amplifier 703.

For example, the power supply 704 powers the piezoelectric disks when configured as transmitters with a 3 MHz signal.

The invention also relates in particular to a method for manufacturing/assembling a device for locating a blood vessel and a corresponding strap.

The invention is not limited to the embodiments of the device for locating a blood vessel and the strap comprising such a device according to the invention shown in the drawings, but stems from an overall view of all the features disclosed herein.

This also applies, in particular, to the potential component assembly. It is therefore conceivable that some of the modules, components or housings described above could be interconnected in one piece.

The invention has been described above on the basis of exemplary embodiments and variants (which may also be referred to as modifications, further developments, alternatives or options). The invention itself is defined by the appended claims. The illustrations and description of the embodiments are provided to explain and understand the claimed invention. Individual features of an exemplary embodiment or its variants can be combined with any other related exemplary embodiment or variant and should also be considered as disclosed in this respect even if they are not expressly described in the context, unless this is clearly impossible or pointless for technical or physical reasons. Conversely, individual features of an exemplary embodiment or its variants do not limit an invention and can be omitted if the remaining combination of features solves a technical problem. In particular, any combination of the individual features described herein that solves a technical problem in a non-obvious way may form a separate subject-matter of the invention.

### LIST OF REFERENCE NUMBERS

1 Locating device
2 acoustic wave transmitter
21 acoustic wave transmitter
22 acoustic wave transmitter
3 acoustic wave receiver
31 acoustic wave receiver
32 acoustic wave receiver
61 first area
62 second area
7 first matrix
8 second matrix
91 first laser
92 second laser
10 strap
11 artery
12 wrist
13 transmitted acoustic waves
14 reflected acoustic waves
100 electronic circuit

1. A device for locating a blood vessel of a limb of a living being, configured to be arranged substantially opposite a skin surface covering the blood vessel of said limb, characterized in that the device comprises at least one acoustic wave transmitter configured to transmit acoustic waves, hereinafter transmitted acoustic waves, in the limb through the skin surface, at least one acoustic wave receiver configured to receive the acoustic waves reflected by the limb, hereinafter reflected acoustic waves, a module for measuring a parameter of the reflected acoustic waves or a difference in value of a parameter between the reflected acoustic waves and the transmitted acoustic waves, and a module for determining the location of the blood vessel depending on said parameter or said difference in value of said parameter.

2. The device according to claim 1, characterized in that the measuring module is a module for measuring the phase shift between the reflected acoustic waves and the transmitted acoustic waves and in that the module for determining the location of the blood vessel takes account of said measured phase shift.

3. The device according to claim 1, characterized in that the measuring module is a module for measuring the amplitude of the reflected acoustic waves and in that the module for determining the location of the blood vessel takes account of said amplitude.

4. The device according to claim 1, characterized in that the measuring module is a module for measuring the difference in frequency between the reflected acoustic waves and the transmitted acoustic waves and in that the module for determining the location of the blood vessel takes account of said difference in frequency.

5. The device according to claim 1, characterized in that it comprises at least one first acoustic wave transmitter and at least one first acoustic wave receiver located in a first area of the device and at least one second acoustic wave transmitter and at least one second acoustic wave receiver located in a second area of the device.

6. The device according to claim 1, characterized in that it comprises a first matrix of acoustic wave transmitters and receivers located in a first area of the device, and a second matrix of acoustic wave transmitters and receivers located in a second area of the device.

7. The device according to claim 5, characterized in that the means for measuring a parameter or a difference in value of a parameter comprise means for measuring a first parameter value or parameter value difference in the first area and means for measuring a second parameter value or parameter value difference in the second area and in that the means for determining the location take account of a first location determined from the first parameter value or parameter value difference and of a second location determined from the second parameter value or parameter value difference.

8. The device according to claim 1, characterized in that each acoustic wave transmitter and/or receiver can be respectively reconfigured as an acoustic wave receiver and/or transmitter.

9. The device according to claim 1, characterized in that the acoustic wave transmitters and receivers comprise piezoelectric elements.

10. The device according to claim 1, characterized in that the module for measuring a parameter or a difference in value of a parameter comprises a filter and an amplifier.

11. The device according to any ene of claim 1, characterized in that it comprises a module for displaying the location of the blood vessel, comprising a first laser and a second laser whose beams intersect at a point on the surface of the skin, said point being indicative of the location of the blood vessel.

12. A strap adapted to be fastened to a limb of a living being characterized in that it comprises the device according to claim 1.
